(19) United States
(12) Patent Application Publication
(10) Pub. No.: US 2006/0058363 A1
(43) Pub. Date: Mar. 16, 2006

(54) Nitric oxide releasing selective cyclooxygenase-2 inhibitors

(76) Inventors: Zhaoyin Wang, Quebec (CA); Robert N Young, Quebec (CA); Robert Zamboni, Quebec (CA)

Correspondence Address: MERCK AND CO., INC, P O BOX 2000, RAHWAY, NJ, 07065-0907, US

(21) Appl. No.: 10/530,214

(22) PCT Filed: Oct. 21, 2003
(86) PCT No.: PCT/CA03/01605

§ 371 Date: Apr. 4, 2005

Related U.S. Application Data
(60) Provisional application No. 60/420,292, filed on Oct. 22, 2002.

(51) Int. Cl. C07D 261/06; C07D 231/12; A61K 31/42; A61K 31/415

(52) U.S. Cl. 514/378; 548/247; 548/377.1; 514/406

(57) ABSTRACT

The invention encompasses novel compounds of Formula (I) and Formula (II), which are nitric oxide-releasing prodrugs useful in the treatment of cyclooxygenase-2 mediated diseases. The invention also encompasses certain pharmaceutical compositions and methods for treatment of cyclooxygenase-2 mediated diseases comprising the use of compounds of Formula (I) or Formula (II). The above compounds may be used as a combination therapy with low-dose aspirin to treat chronic cyclooxygenase-2 mediated diseases or conditions while simultaneously reducing the risk of thrombotic cardiovascular events.

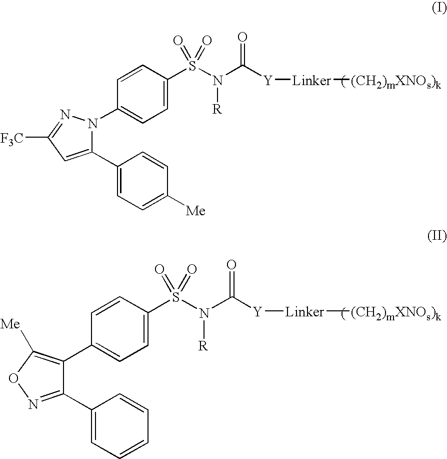

### BACKGROUND OF THE INVENTION

Selective inhibitors of cyclooxygenase-2 are a sub-class of the class of drugs known as non-steroidal antiinflammatory drugs (NSAIDs). The NSAIDs are active in reducing the prostaglandin-induced pain and swelling associated with the inflammation process but are also active in affecting other prostaglandin-regulated processes not associated with the inflammation process. Thus, use of high doses of most common NSAIDs can produce severe side effects, including life threatening ulcers, that limit their therapeutic potential. An alternative to NSAIDs is the use of corticosteroids, which have even more drastic side effects, especially when long term therapy is involved.

Previous NSAIDs have been found to prevent the production of prostaglandin by inhibiting enzymes in the human arachidonic acid/prostaglandin pathway including the enzyme cyclooxygenase (COX). The discovery that there are two isoforms of the COX enzyme, the first, COX-1, being involved with physiological functions and the second, COX-2, being induced in inflamed tissue, has given rise to a new approach. While conventional NSAIDs block both forms of the enzyme, the identification of the inducible COX-2 enzyme associated with inflammation has provided a viable target of inhibition which more effectively reduces inflammation and produces fewer and less drastic side effects. Many compounds which have activity as COX-2 inhibitors have been identified, including rofecoxib (VIOXX®), etoricoxib (ARCOXIA™), celecoxib (CELEBREX®) and valdecoxib (BEXTRA™), and much research continues in this area.

Many patients with a chronic cyclooxygenase-2 mediated disease or condition are elderly and thus are at increased risk for thrombotic cardiovascular events, such as stroke, myocardial ischemia, myocardial infarction, angina pectoris, transient ischemic attack (TIA; amaurosis fugax), reversible ischemic neurologic deficits, and any similar thrombotic event in any vascular bed (splanchnic, renal, aortic, peripheral, etc.). Moreover, there is evidence that patients with chronic inflammatory conditions, such as rheumatoid arthritis and systemic lupus erythematosis are at increased risk for thrombotic cardiovascular events. Thus, it is desirable that such patients receive appropriate therapy to reduce their risk of such events.

NO-releasing forms of non-steroidal anti-inflammatory drugs are known in the art and are reported to have improved gastrointestinal and cardiovascular safety profiles over their conventional NSAID counterparts. Furthermore, NO-releasing forms of selective cyclooxygenase-2 selective inhibitors are disclosed in WO 01/45703, published on Jun. 28, 2001, which is hereby incorporated by reference in its entirety.

The present invention provides for novel nitrosated or nitrosylated prodrugs for cyxlooxygenase-2 selective inhibitors that are useful for treating cyclooxygenase-2 mediated diseases or conditions which can be administered alone or in combination with low-dose aspirin. Thus, the invention provides for a clearly superior profile than that hitherto obtainable in that it provides efficacy in treating chronic cyclooxygenase-2 mediated diseases or conditions, effectively reducing the risk of thrombotic cardiovascular events and renal side effects and at the same time reduces the risk of GI ulceration or bleeding.

In particular, the present invention provides novel prodrugs of celebrex (Compound A) and Valdecoxib (Compound B): 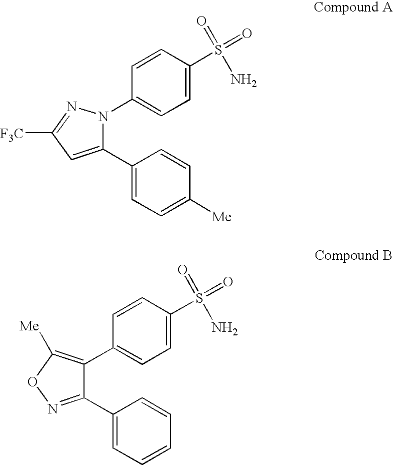
Celebrex, methods of preparing celecoxib and methods of using celecoxib are disclosed in U.S. Pat. No. 5,466,823, issued Nov. 14, 1995, which is hereby incorporated by reference. Valdecoxib, methods of preparing valdecoxib and methods of using valdecoxib are disclosed in U.S. Pat. No. 5,633,272, issued May 27, 1997, which is hereby incorporated by reference.

### SUMMARY OF THE INVENTION

The invention encompasses novel compounds of Formula I and Formula II, which are nitric oxide-releasing prodrugs useful in the treatment of cyclooxygenase-2 mediated diseases.

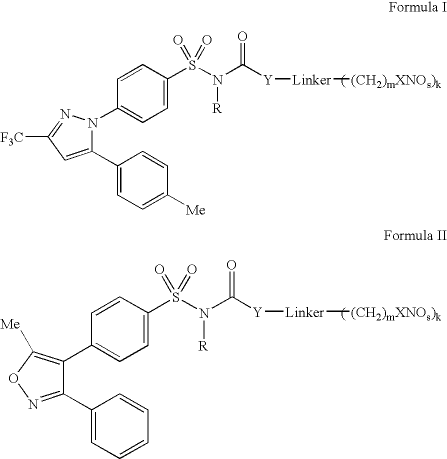

The invention also encompasses certain pharmaceutical compositions and methods for treatment of cyclooxygenase-2 mediated diseases comprising the use of compounds of Formula I or Formula II. The above compounds may be used as a combination therapy with low-dose aspirin to treat chronic cyclooxygenase-2 mediated diseases or conditions while simultaneously reducing the risk of thrombotic cardiovascular events.

### DETAILED DESCRIPTION OF THE INVENTION

The invention encompasses novel compounds of Formula I and Formula II, which are nitric oxide-releasing prodrugs useful in the treatment of cyclooxygenase-2 mediated diseases. 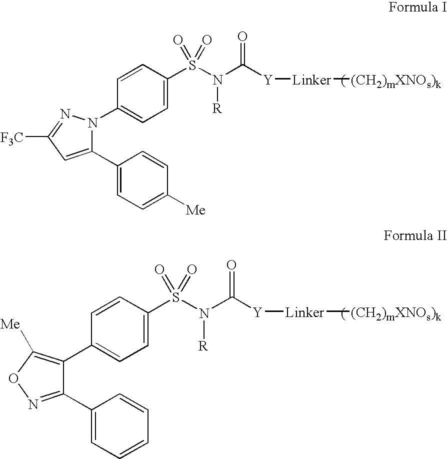
or a pharmaceutically acceptable salt thereof wherein
each s is independently 1 or 2;
k is 1, 2, 3 or 4;
each m is independently 0, 1, 2, 3 or 4;
each X is independently O or S;
Y is a bond, S, O or NR₁, wherein R1 is hydrogen or C₁₋₆alkyl;
R is hydrogen or C₁₋₆alkyl;
the Linker is selected from the group consisting of:
   (a) —(CH₂)ₙ, wherein n is 0, 1, 2, 3 or 4,
   (b) C₃₋₆cycloalkyl, wherein the C₃₋₆cycloalkyl optionally mono-, di- or tri-substituted with a substituent selected from the group consisting of (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkthio,
   (c) aryl, wherein the aryl is selected from the group consisting of phenyl and naphthyl, wherein the aryl is optionally mono-, di- or tri-substituted with a substituent selected from the group consisting of
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkthio,
   (d) Heteroaryl optionally mono-, di- or tri-substituted with substituents selected from the group consisting of,
      (1) halo,
      (2) C₁₋₃alkyl,
      (3) C₁₋₃alkoxy,
      (4) Hydroxy,
      (5) NO₂,
      (6) CO₂,
      (7) CF₃,
      (8) CN;
      (9) CH₂COOH
      (10) CH₂COO—C₁₋₃alkyl,
      (11) C₁₋₃alkthio.
         For purposes of this specification, heteroaryl or benzoheteroaryl group includes benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl.

The present compounds are nitric oxide releasing prodrugs which liberate nitric oxide and celecoxib or valdecoxib in vivo and can be administered alone or in combination with low dose aspirin. Thus, the invention provides for a clearly superior profile than that hitherto obtainable in that it provides efficacy in treating chronic cyclooxygenase-2 mediated diseases or conditions, effectively reducing the risk of thrombotic cardiovascular events and renal side effects and at the same time reduces the risk of GI ulceration or bleeding.

An embodiment of the invention encompasses a compound of Formula I and Formula II wherein —S(O)₂NH₂ is replaced with S(O)₂CH₃.

Within this embodiment of the invention is encompassed a compound of Formula I wherein:
s is 2;
k is 1;
m is 1 or 2.

Also this embodiment of the invention is encompassed a compound of Formula I wherein R is hydrogen.

Also this embodiment of the invention is encompassed a compound of Formula I wherein X is O.

Also this embodiment of the invention is encompassed a compound of Formula I wherein Y is a bond.

Also this embodiment of the invention is encompassed a compound of Formula I wherein k is 1.

Also this embodiment of the invention is encompassed a compound of Formula I wherein m is 1.

Also this embodiment of the invention is encompassed a compound of Formula I wherein
R is hydrogen;
Y is a bond;
s is 2;
k is 1; and
m is 1.

Also within this embodiment of the invention is encompassed a compound of Formula I wherein the Linker is —(CH₂)ₙ, wherein n is 1 or 2.

Also within this embodiment of the invention is encompassed a compound of Formula I wherein the Linker is C₃₋₆cycloalkyl, wherein the C₃₋₆cycloalkyl optionally mono-, di- or tri-substituted with a substituent selected from the group consisting of
(1) halo,
(2) Methyl,
(3) Methoxy,
(4) Hydroxy,
(5) NO₂,
(6) CO₂,
(7) CF₃,
(8) CN;
(9) CH₂COOH

Also within this embodiment of the invention is encompassed a compound of Formula I wherein the Linker is aryl, wherein the aryl is selected from the group consisting of phenyl and naphthyl, wherein the aryl is optionally mono-, di- or tri-substituted with a substituent selected from the group consisting of
(1) halo,
(2) Methyl,
(3) Methoxy,
(4) Hydroxy,
(5) NO₂,
(6) CO₂,
(7) CF₃,
(8) CN,
(9) CH₂COOH.

Also within this embodiment of the invention is encompassed a compound of Formula I and Formula II wherein the Linker is benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, optionally mono-, di- or tri-substituted with substituents selected from the group consisting of,
(1) halo,
(2) Methyl,
(3) Methoxy,
(4) Hydroxy,
(5) NO₂,
(6) CO₂,
(7) CF₃,
(8) CN;
(9) CH₂COOH

Another embodiment of the invention encompasses the compound of Formula I or II wherein the Linker is pyridyl optionally substituted as above.

Another embodiment of the invention encompasses a compound of Formula I or Formula II wherein s is 2.

Another embodiment of the invention encompasses a compound selected from the following group:

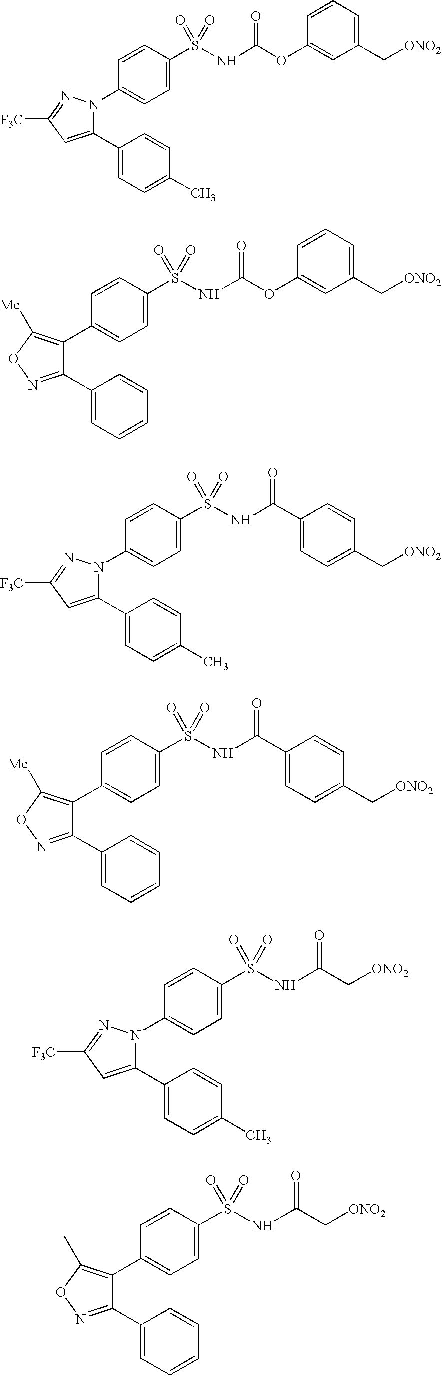

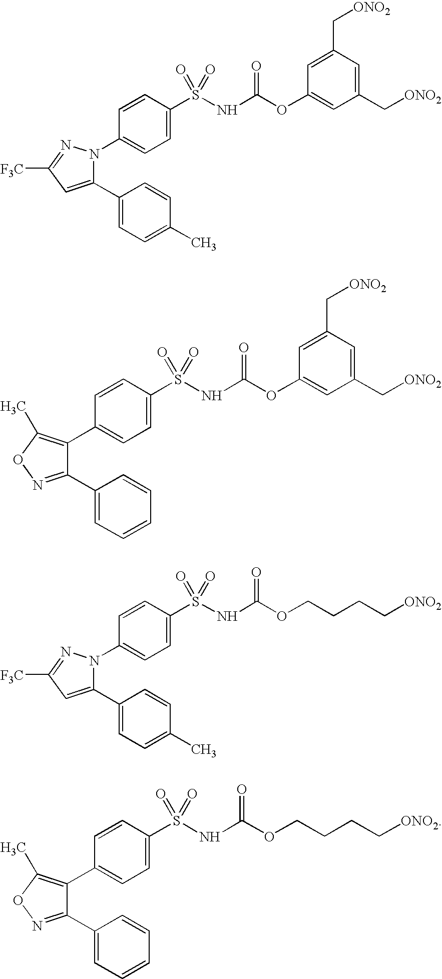

Another embodiment of the invention encompasses a compound of Formula I or Formula II 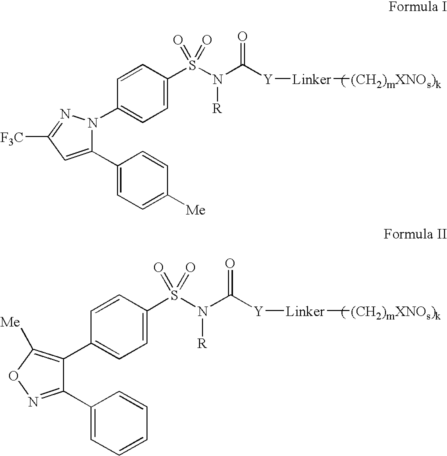
or a pharmaceutically acceptable salt thereof wherein
each s is independently 1 or 2;
k is 1, 2, 3 or 4;
each m is independently 0, 1, 2, 3 or 4;
each X is independently O or S;
Y is a bond, S, O or NR₁, wherein R₁ is hydrogen or C₁₋₆alkyl;
R is hydrogen or C₁₋₆alkyl;
the Linker is selected from the group consisting of:

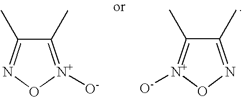

The invention also encompasses a pharmaceutical composition comprising a compound of Formula I or Formlua II and a pharmaceutically acceptable carrier.

The invention also encompasses a method of treating an inflammatory disease susceptible to treatment with a non-steroidal anti-inflammatory agent comprising administering to a patient in need of such treatment of a non-toxic therapeutically effective amount of a compound of Formula I or Formula II. Within this embdiment is encompassed the above method wherein the patient is also at risk of a thrombotic cardiovascular event.

Another embodiment of the invention encompasses method of treating cyclooxygenase mediated diseases advantageously treated by an active agent that selectively inhibits COX-2 in preference to COX-1 comprising administering to a patient in need of such treatment of a non-toxic therapeutically effective amount of a compound of Formula I. Within this embodiment is encompassed the above method wherein the patient is also at risk of a thrombotic cardiovascular event.

Another embodiment of the invention encompasses a method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a compound of Formula I in an amount effective to treat the cyclooxygenase-2 mediated disease or condition and aspirin in an amount effective to reduce the risk of the thrombotic cardiovascular event. Within this embodiment is encompassed the above method wherein the compound of Formula I is administered orally on a once daily basis. Within this embodiment is encompassed the above method wherein the compound of Formula I or Formula II is administered orally on a twice daily basis. Within this embodiment is encompassed the above method wherein the cyclooxygenase-2 selective mediated disease or condition is selected from the group consisting of: osteoarthritis, rheumatoid arthritis and chronic pain. Within this embodiment is encompassed the above method wherein aspirin is administered at a dose of about 30 mg to about 1 g. Within this embodiment is encompassed the above method wherein aspirin is administered at a dose of about 80 to about 650 mg. Within this embodiment is encompassed the above method wherein aspirin is administered at a dose of about 81 mg or about 325 mg. Within this embodiment is encompassed the above method wherein aspirin is orally administered once daily.

The invention also encompasses a pharmaceutical composition comprising a compound of Formula I or Formlua II and aspirin in combination with a pharmaceutically acceptable carrier.

For purposes of this specification alkyl is defined to include linear, branched, and cyclic structures, with C₁₋₆alkyl including including methyl, ethyl, propyl, 2-propyl, s- and t-butyl, butyl, pentyl, hexyl, 1,1-dimethylethyl, cyclopropyl, cyclobutyl, cyclopentyl and cyclohexyl. Similarly, C₁₋₆alkoxy is intended to include alkoxy groups of from 1 to 6 carbon atoms of a straight, branched, or cyclic configuration. Examples of lower alkoxy groups include methoxy, ethoxy, propoxy, isopropoxy, cyclopropyloxy, cyclohexyloxy, and the like. Likewise, C₁₋₆alkylthio is intended to include alkylthio groups of from 1 to 6 carbon atoms of a straight, branched or cyclic configuration. Examples of lower alkylthio groups include methylthio, propylthio, isopropylthio, cycloheptylthio, etc. By way of illustration, the propylthio group signifies —SCH₂CH₂CH₃.

Some of the compounds described herein contain olefinic double bonds, and unless specified otherwise, are meant to include both E and Z geometric isomers.

The term “treating a chronic cylcooxygenase-2 mediated disease or condition” means treating or preventing any chronic disease or condition that is advantageously treated or prevented by inhibiting the cyclooxygenase-2 enzyme. The term includes the relief of pain, fever and inflammation of a variety of conditions including rheumatic fever, symptoms associated with influenza or other viral infections, common cold, low back pain, neck pain, dysmenorrhea, headache, migraine, toothache, sprains and strains, myositis, neuralgia, synovitis, arthritis, including rheumatoid arthritis, degenerative joint diseases (osteoarthritis), gout, ankylosing spondylitis, bursitis, burns, injuries, and pain and inflammation following surgical procedures. In addition, such a compound may inhibit cellular neoplastic transformations and metastatic tumor growth and hence can be used in the treatment and/or prevention of cancer. In addition, such a compound may inhibit the onset or progression of Altzheimer's disease or cognitive impairment. The term also includes the treatment and/or prevention of cyclooxygenase-mediated proliferative disorders such as may occur in diabetic retinopathy and tumor angiogenesis. The term “treating” encompasses not only treating a patient to relieve the patient of the signs and symptoms of the disease or condition but also prophylactically treating an asymptomatic patient to prevent the onset or progression of the disease or condition.

A “thrombotic cardiovascular event” is defined as any sudden event of a type known to be caused by platelet aggregation, thrombosis, and subsequent ischemic clinical events, including thrombotic or thromboembolic stroke, myocardial ischemia, myocardial infarction, angina pectoris, transient ischemic attack (TIA; amaurosis fugax), reversible ischemic neurologic deficits, and any similar thrombotic event in any vascular bed (splanchnic, renal, aortic, peripheral, etc.).

The term “patient in need of such treatment and at risk of a thrombotic cardiovascular event” means a patient in need of both treatment for a cyclooxygenase-2 mediated disease and also at risk of a thrombotic cardiovascular event. One skilled in the art can diagnose a patient that is in need of treatment for a cyclooxygenase-2 mediated disease or condition and also at risk of suffering a thrombotic cardiovascular event. For example, such a patient may be over the age of 50 with osteoarthritis and with a previous myocardial infarction. Other risk factors for a thrombotic cardiovascular event include hypertension, hypercholesterolemia, diabetes mellitus, chronic renal impairment, smoking, and any prior personal or family history of such an event. Administration of the drug combination to the patient includes both self-administration and administration to the patient by another person.

The terms “nitric oxide releasing-cyclooxygenase-2 selective inhibitor,” “NO-cyclooxygenase-2 selective inhibitor,” “nitric oxide releasing-COX-2 inhibitor” and “NO-COX-2 inhibitor” mean a modified version of a cycloxygenase-2 selective inhibitor or a prodrug as defined above linked to a NO releasing moiety by means of a linking group such as an ester linkage.

The term “amounts that are effective to treat” is intended to mean that amount of a drug or pharmaceutical agent that will elicit the biological or medical response of a tissue, a system, animal or human that is being sought by a researcher, veterinarian, medical doctor or other clinician. The term also encompasses the amount of a pharmaceutical drug that will prevent or reduce the risk of occurrence of the biological or medical event that is sought to be prevented in a tissue, a system, animal or human by a researcher, veterinarian, medical doctor or other clinician. The inhibitor of cyclooxygenase-2 may be administered at a dosage level up to conventional dosage levels for NSAIDs. Suitable dosage levels will depend upon the antiinflammatory effect of the chosen inhibitor of cyclooxygenase-2, but typically suitable levels will be about 0.01 to about 50 mg/kg per day. The compound may be administered on a regimen of once or twice per day.

The term “amount effective to reduce the risk of” means the amount of a pharmaceutical drug that will prevent or reduce the risk of occurrence of the biological or medical event that is sought to be prevented in a tissue, a system, animal or human by a researcher, veterinarian, medical doctor or other clinician. Aspirin is administered at a dose of about 30 mg to about 1 g once daily, preferably at a dose of about 80 mg to about 650 mg.

The term “concomitantly administering” means administering the agents substantially concurrently. The term “concomitantly administering” encompasses not only administering the two agents in a single pharmaceutical dosage form but also the administration of each active agent in its own separate pharmaceutical dosage formulation. Where separate dosage formulations are used, the agents can be administered at essentially the same time, i.e., concurrently.

The term “sequentially administering” means administering the agents at separately staggered times. Thus, agents can be sequentially administered such that the beneficial pharmaceutical effect of NO-aspirin and the COX-2 inhibitor or aspirin and the NO-COX-2 inhibitor are realized by the patient at substantially the same time. Thus, for example, if a COX-2 selective inhibitor and NO releasing aspirin are both administered on a once a day basis, the interval of separation between sequential administration of the two agents can be up to twelve hours apart.

The pharmaceutical compositions of the present invention comprise a compound of Formula I as an active ingredient or a pharmaceutically acceptable salt, thereof, and may also contain a pharmaceutically acceptable carrier and optionally other therapeutic ingredients. The term “pharmaceutically acceptable salts” refers to salts prepared from pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

It will be understood that in the discussion of methods of treatment which follows, references to the compounds of Formula I or Formula II are meant to also include the pharmaceutically acceptable salts.

The Compound of Formula I and Formula II is useful for the relief of pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, following surgical and dental procedures. In addition, such a compound may inhibit cellular neoplastic transformations and metastic tumor growth and hence can be used in the treatment of cancer. Compounds of Formula I may also be useful for the treatment of dementia including pre-senile and senile dementia, and in particular, dementia associated with Alzheimer Disease (i.e. Alzheimer's dementia).

Compounds of Formula I and Formula II will also inhibit prostanoid-induced smooth muscle contraction by preventing the synthesis of contractile prostanoids and hence may be of use in the treatment of dysmenorrhea, premature labor and asthma. They will also be useful to inhibit bone loss (osteoporosis).

By virtue of its high cyclooxygenase-2 (COX-2) activity and/or its selectivity for cyclooxygenase-2 over cyclooxygenase-1 (COX-1) as defined above, compounds of Formula I and Formula II will prove useful as an alternative to conventional non-steroidal antiinflammatory drugs (NSAID'S) particularly where such non-steroidal antinflammatory drugs may be contra-indicated such as in patients with peptic ulcers, gastritis, regional enteritis, ulcerative colitis, diverticulitis or with a recurrent history of gastrointestinal lesions; GI bleeding, coagulation disorders including anemia such as hypoprothrombinemia, haemophilia or other bleeding problems (including those relating to reduced or impaired platelet function); kidney disease (e.g. impaired renal function); those prior to surgery or taking anticoagulants; and those susceptible to NSAID induced asthma.

Similarly, compounds of Formula I and Formula II, will be useful as a partial or complete substitute for conventional NSAID'S in preparations wherein they are presently co-administered with other agents or ingredients. Thus in further aspects, the invention encompasses pharmaceutical compositions for treating cyclooxygenase-2 mediated diseases as defined above comprising a non-toxic therapeutically effective amount of the compound of Formula I or Formula II as defined above and one or more ingredients such as another pain reliever including acetominophen or phenacetin; a potentiator including caffeine; an H2-antagonist, aluminum or magnesium hydroxide, simethicone, a decongestant including phenylephrine, phenylpropanolamine, pseudophedrine, oxymetazoline, ephinephrine, naphazoline, xylometazoline, propylhexedrine, or levo-desoxyephedrine; an antiitussive including codeine, hydrocodone, caramiphen, carbetapentane, or dextramethorphan; a diuretic; a sedating or non-sedating antihistamine. In addition the invention encompasses a method of treating cyclooxygenase mediated diseases comprising: administration to a patient in need of such treatment a non-toxic therapeutically effect amount of the compound of Formula I or Formula II, optionally co-administered with one or more of such ingredients as listed immediately above.

Compounds of the present invention are inhibitors of cyclooxygenase-2 and are thereby useful in the treatment of cyclooxygenase-2 mediated diseases as enumerated above. This activity is illustrated by their ability to selectively inhibit cyclooxygenase-2 over cyclooxygenase-1. Accordingly, in one assay, the ability of the compounds of this invention to treat cyclooxygenase mediated diseases can be demonstrated by measuring the amount of prostaglandin E₂ (PGE₂) synthesized in the presence of arachidonic acid, cyclooxygenase-1 or cyclooxygenase-2 and a compound of Formula I. The IC₅₀ values represent the concentration of inhibitor required to return PGE₂ synthesis to 50% of that obtained as compared to the uninhibited control. For the treatment of any of these cyclooxygenase mediated diseases, compounds of Formula I and Formula II may be administered orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques. In addition to the treatment of warm-blooded animals such as mice, rats, horses, cattle sheep, dogs, cats, etc., the compound of the invention is effective in the treatment of humans.

As indicated above, pharmaceutical compositions for treating cyclooxygenase-2 mediated diseases as defined may optionally include one or more ingredients as listed above.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or an oil medium, for example peanut oil, liquid paraffin, or olive oil.

Aqueous suspensions contain the active material in admixture with excipients suitable for the manufacture of aqueous suspensions. Such excipients are suspending agents, for example sodium carboxymethyl-cellulose, methylcellulose, hydroxypropylmethy-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of an oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds of Formula I may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compound of Formula I or Formula II are employed. (For purposes of this application, topical application shall include mouth washes and gargles.)

Dosage levels of the order of from about 0.01 mg to about 50 mg/kg of body weight per day are useful in the treatment of the above-indicated conditions, or alternatively about 0.5 mg to about 2 g per patient per day. For example, inflammation may be effectively treated by the administration of from about 0.01 to 50 mg of the compound per kilogram of body weight per day, or alternatively about 0.5 mg to about 2 g per patient per day.

The amount of active ingredient that may be combined with the carrier materials to produce a single dosage form will vary depending upon the host treated and the particular mode of administration. For example, a formulation intended for the oral administration of humans may contain from 0.5 mg to 2 g of active agent compounded with an appropriate and convenient amount of carrier material which may vary from about 5 to about 95 percent of the total composition. Dosage unit forms will generally contain between from about 1 mg to about 500 mg of an active ingredient, typically 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

Methods of Synthesis

Method A

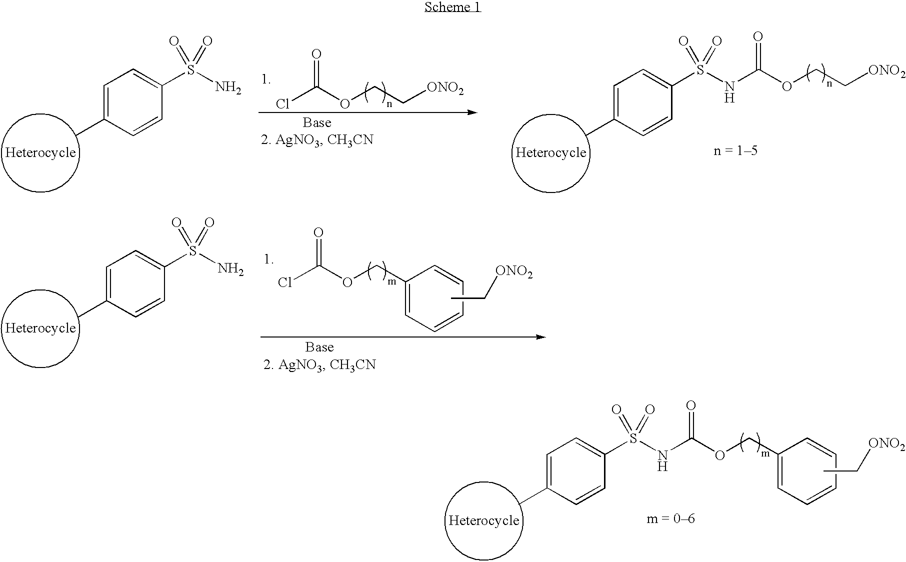

The sulfonylcarbamate derivatives can be prepared according to Scheme 1. Reaction of celecoxib or valdecoxib can react with a suitable chlorocarbonate and a base to give the desired product. Alternatively, the desired sulfonylcarbamates can be prepared by converting celecoxib or valdecoxib to bromide derivatives and then nitration of the bromides with silver nitrate (Scheme 2). 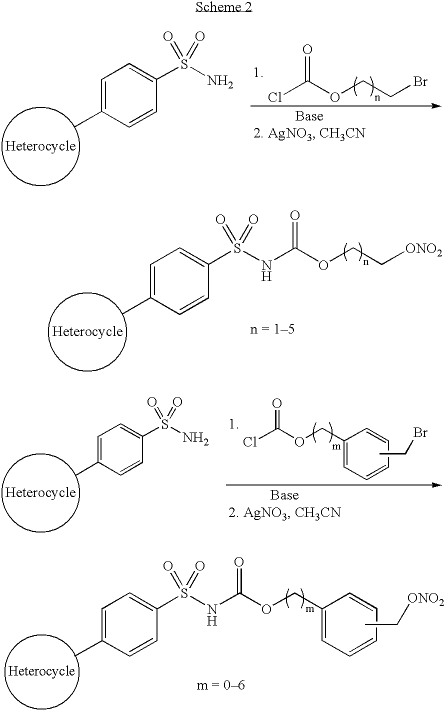
Method B

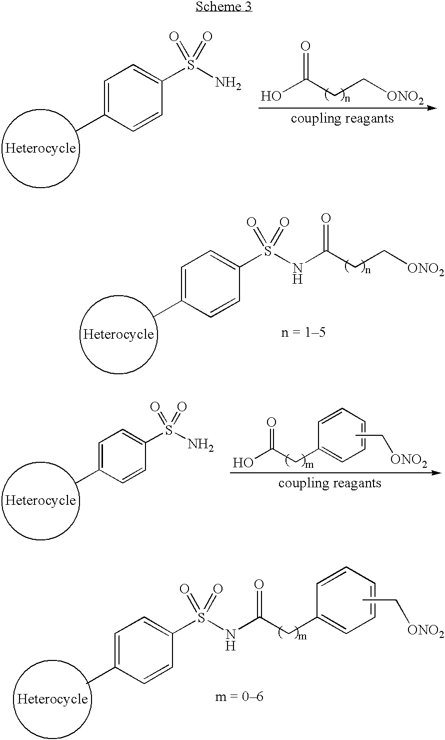

The acylsulfonamide derivatives can be prepared by reaction of celecoxib or valdecoxib with the appropriate nitrate containing carboxylic acids (Scheme 3) and a coupling reagent. The standard coupling reagents such as DCC, ECDI, CMC, carbonyldiimidazole or oxalyl chloride can be used for this reaction. Alternatively, celecoxib or valdecoxib can be coupled with the appropriately protected hydroxy-acid derivatives. The resulting intermediates can be deprotected and converted to the corresponding bromides, followed by nitration with silver nitrate to give the desired products (Scheme 4). 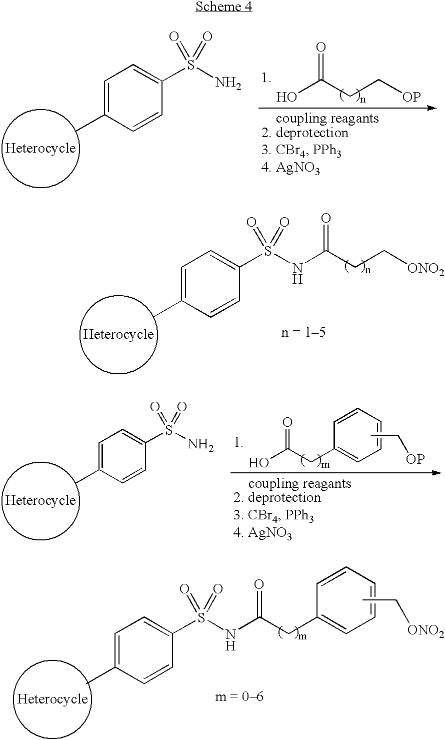
Assays for Determining Biological Activity

The compound of Formula I can be tested using the following assays to determine their biological activity.

Inhibition of Cyclooxygenase Activity

Compounds are tested as inhibitors of cyclooxygenase activity in whole cell and microsomal cyclooxygenase assays. Both of these assays measure prostaglandin E₂ (PGE₂) synthesis in response to arachidonic acid, using a radioimmunoassay. Cells used for whole cell assays, and from which microsomes are prepared for microsomal assays, are human osteosarcoma 143 cells (which specifically express cyclooxygenase-2) and human U-937 cells (which specifically express cyclooxygenase-1). In these assays, 100% activity is defined as the difference between prostaglandin E₂ synthesis in the absence and presence of arachidonate addition. IC₅₀ values represent the concentration of putative inhibitor required to return PGE₂ synthesis to 50% of that obtained as compared to the uninhibited control.

Representative Rat Paw Edema Assay—Protocol

Male Sprague-Dawley rats (150-200 g) are fasted overnight and are given p.o., either vehicle (1% methocell) or a test compound in the morning. One hr later, a line is drawn using a permanent marker at the level above the ankle in one hind paw to define the area of the paw to be monitored. The paw volume (V_{Oh}) is measured using a plethysmometer (Ugo-Basile, Italy) based on the principle of water displacement. The animals are then injected subplantarly with 50 ul of a 1% carrageenan solution in saline (Sigma Chem) into the paw using an insulin syringe with a 25-gauge needle (i.e. 500 ug carrageenan per paw). Three hr later, the paw volume (V₃ₕ) is measured and the increases in paw volume (V₃ₕ−V_{Oh}) are calculated. Paw edema data are compared with the vehicle-control group and percent inhibition calculated taking the values in the control group as 100%. All treatment groups are coded to eliminate observer bias.

NSAID-Induced Gastropathy in Rats

Rationale

The major side effect of conventional NSAIDs is their ability to produce gastric lesions in man. Rats are sensitive to the actions of NSAIDs and have been used commonly in the past to evaluate the gastrointestinal side effects of current conventional NSAIDs. In the present assay, NSAID-induced gastrointestinal damage is observed by measuring urinary ⁵¹Cr excretion after oral dosing of ⁵¹Cr-EDTA. Urinary ⁵¹Cr excretion is a well-established and sensitive technique to detect gastrointestinal integrity in animals and man.

Methods

Male Sprague-Dawley rats (150-200 g) are administered orally a test compound either once (acute dosing) or in multiple doses for a few days (chronic dosing). Immediately after the administration of the last dose, the rats are given an oral dose of ⁵¹Cr-EDTA (10 μCi/rat). The animals are placed individually in metabolism cages with food and water ad lib. Urine is collected for a 24 hr period and ⁵¹Cr urinary excretion is calculated as a percent of total ingested dose.

Protein-Losing Gastrophathy in Squirrel Monkeys

Rationale

Protein-losing gastropathy (manifested as appearance of circulating cells and plasma proteins in the GI tract) is a significant and dose-limiting adverse response to NSAIDs. This can be quantitatively assessed by intravenous administration or ⁵¹CrCl₃ solution. This isotopic ion can avidly bind to cell and serum globins and cell endoplasmic reticulum. Measurement of radioactivity appearing in feces collected for 24 hr after administration of the isotope thus provides a sensitive and quantitative index of protein-losing gastropathy.

Methods

Groups of male squirrel monkeys (0.8 to 1.4 kg) are treated by gavage with 1% methocel or a test compounds at multiple doses for a few days. Intravenous ⁵¹Cr (5 μCi/kg in 1 ml/kg PBS) is administered 1 hr after the last drug/vehicle dose, and feces collected for 24 hr in a metabolism cage and assessed for excreted ⁵¹Cr by gamma-counting. ⁵¹Cr fecal excretion is calculated as a percent of total injected dose.

Rat Aortic Smooth Muscle Rings in Male Spargue-Dawley Rats

Preparation of rat aortic smooth muscle rings Male Sprague-Dawley rats (Charles River Laboratories (Wilmington, Mass.) were euthanized by intraperiton injection of a high dose of sodium pentobarbitone (80-100 mg/kg). The thoracic aorta was rapidly excised and immediately placed in a Petri dish containing warm (37° C.) oxygenated (95% 0, and 5% CO2) Kreb's buffer (composition per millimolar: NaCl (119); KCI (4.69); CaCl₂.H₂O (2.52); MgSO₄.7H₂O (0.57); NaHCO₂, (25); NaH₂PO.,.H₂O (1.01) and glucose (11.1). Under a stereoscopic dissecting microscope, the aorta was cleaned, freed from adhering fat and connective tissues. The tissue was cut into ring segments, each approximately 2-3 mm in length.

For experiments to measure relaxation of the tissue under various conditions, a stainless steel tissue holder and an U-shaped stainless steel wire were inserted into the lumen of the aortic ring. The tissue holder anchored the ring at 142 the bottom of the organ bath whereas the end of the U-shaped steel wire was tied with fine silk thread so that it connected to the FT-202 transducer. The tissue holder and the steel wire along with the aortic ring were then suspended in a 5-ml, double-jacketed temperature-controlled glass organ bath (Radnoti Glass Technology, Inc., Monrovia, Calif.) filled with fresh Kreb's buffer. A mixture of 95% O₂ and 5% CO₂ was bubbled through a porous sintered disc at the bottom of the bath. The rings were given an initial resting tension of 1.5 g and the preparation was allowed to equilibrate at the initial tension for about 90 minutes. During this equilibration period, the bath fluid was changed every 15 minutes and replaced with fresh prewarmed (37° C.) Kreb's buffer. The isometric tension of the aortic muscle at rest and its response to different stimuli were recorded on a Power Macintosh 6100 computer via a MacLab 8/S computer interface (CB Sciences, Inc, Milford, Mass.) after an initial amplification through a low-noise ETH-400 bioamplifier (CB Sciences, Inc, Milford, Mass.). Contractile responsiveness of the tissue strips was established with 10 jaM phenylephrine, and the strips were incubated with the drug for 20 minutes to establish a steady level of contraction.

To test the relaxation effects, test compounds were added to the phenylephrine precontracted strips in the tissue bath at cumulative concentrations of 0.1 μM to 0.1 mM. Concentration of test compounds was increased only after relaxation at the previous concentration had reached a plateau level.

### REPRESENTATIVE EXAMPLES

The invention will now be illustrated by the following non-limiting examples in which, unless stated otherwise:
(i) all operations were carried out at room or ambient temperature, that is, at a temperature in the range 18-25° C.; evaporation of solvent was carried out using a rotary evaporator under reduced pressure (600-4000 pascals: 4.5-30 mm. Hg) with a bath temperature of up to 60° C.; the course of reactions was followed by thin layer chromatography (TLC) and reaction times are given for illustration only; melting points are uncorrected and ‘d’ indicates decomposition; the melting points given are those obtained for the materials prepared as described; polymorphism may result in isolation of materials with different melting points in some preparations; the structure and purity of all final products were assured by at least one of the following techniques: TLC, mass spectrometry, nuclear magnetic resonance (NMR) spectrometry or microanalytical data; yields are given for illustration only; when given, NMR data is in the form of delta (δ) values for major diagnostic protons, given in parts per million (ppm) relative to tetramethylsilane (TMS) as internal standard, determined at 300 MHz or 400 MHz using the indicated solvent; conventional abbreviations used for signal shape are: s. singlet; d. doublet; t. triplet; m. multiplet; br. broad; etc.: in addition “Ar” signifies an aromatic signal; chemical symbols have their usual meanings; the following abbreviations have also been used v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

The following abbreviations have the indicated meanings:
Ac=Acetyl
Bn=Benzyl
DBU=1,8-diazabicyclo[5.4.0]undec-7-ene
DIBAL=diisobutylaluminum hydride
DMAP=4-(dimethylamino)pyridine
DMF=N,N-dimethylformamide
Et₃N=Triethylamine
HBSS=Hanks' balanced salt solution
LDA=lithium diisopropylamide
m-CPBA=Metachloroperbenzoic acid
MMPP=monoperoxyphtalic acid
MPPM=monoperoxyphthalic acid, magnesium salt 6H₂O
Ms=methanesulfonyl=mesyl=S(O)₂Me
Ms0=methanesulfonate=mesylate
NSAID=non-steroidal anti-inflammatory drug
OXONE®=2KHSO₅.KHSO₄.K₂SO₄
PBS=phosphate buffered saline
PCC=pyridinium chlorochromate
PDC=pyridinium dichromate
Ph=Phenyl
Phe=Benzenediyl
Pye=Pyridinediyl
r.t.=room temperature
rac.=Racemic
SAM=aminosulfonyl or sulfonamide or S(O)₂NH₂
TBAF=tetra-n-butylammonium fluoride
Th=2- or 3-thienyl
TFAA=trifluoroacetic acid anhydride
THF=Tetrahydrofuran
Thi=Thiophenediyl
TLC=thin layer chromatography
TMS-CN=trimethylsilyl cyanide
Tz=1H (or 2H)-tetrazol-5-yl
C₃H₅=Allyl
   Alkyl Group Abbreviations
Me=Methyl
Et=Ethyl
n-Pr=normal propyl
i-Pr=Isopropyl
n-Bu=normal butyl
i-Bu=Isobutyl
s-Bu=secondary butyl
t-Bu=tertiary butyl
c-Pr=Cyclopropyl
c-Bu=Cyclobutyl
c-Pen=Cyclopentyl
c-Hex=Cyclohexyl

### Example 1

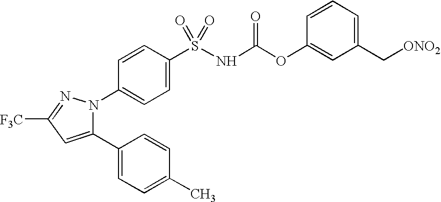

### 4-[(NITROOXY)METHYL]PHENYL {4-[5-(4-METHYLPHENYL)-3-(TRIFLUOROMETHYL)-1H-PYRAZOL-1-YL]PHENYL}SULFONYLCARBAMATE

To a solution of 3-nitrooxymethylphenol (1.69 g, 10 mmol) and triphosgene (0.92 g, 3.1 mmol) in 100 ml of CH₂Cl₂, pyridine (0.86 g, 11 mmol) is added dropwise at −78° C. After 1 h of stirring at rt, the final suspension is transferred via a canula to a solution of Celebrex (3.81 g, 10 mmol) and pyridine 0.86 g in CH₂Cl₂ cooled at −78° C. After 1 h at 0° C., the reaction mixture is poured into a separatory funnel containing EtOAc(50 mL)/NH₄Cl(50 mL, sat). The phases are separated and the aqueous phase is extracted twice with EtOAc (2×50 mL). The organic layers are combined, washed with brine, dried over anhydrous Na₂SO₄ and evaporated to dryness. The crude material can be further purified by flash chromatography eluting with 1:3 EtOAc/hexanes to yield the title compound.

### Example 2

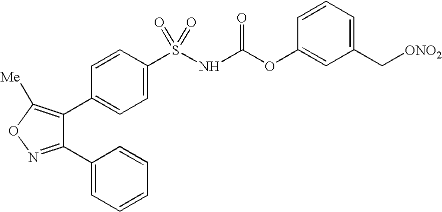

### 4-[(NITROOXY)METHYL]PHENYL [4-(5-METHYL-3-PHENYLISOXAZOL-4-YL)PHENYL]SULFONYLCARBAMATE

Starting from valdecoxib, the title compound is prepared by following the same conditions as described in Example 1.

### Example 3

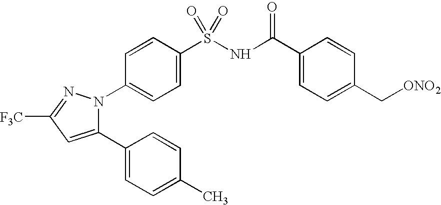

### 4-{[({4-[5-(4-METHYLPHENYL)-3-(TRIFLUOROMETHYL)-1H-PYRAZOL-1-YL]PHENYL}SULFONYL)AMINO]CARBONYL}BENZYL NITRATE

To a solution of 4-nitrooxymethylbenzoic acid (1.97 g, 10 mmol) and Celebrex (3.81 g, 10 mmol) in 100 ml of CH₂Cl₂ is added EDCI (3.8 g, 20 mmol) at 0° C. After 1 h of stirring at rt, the reaction mixture is poured into a separatory funnel containing EtOAc(50 mL)/NH₄Cl(50 mL, sat). The phases are separated and the aqueous phase is extracted twice with EtOAc (2×50 mL). The organic layers are combined, washed with brine, dried over anhydrous Na₂SO₄ and evaporated to dryness. The crude material can be further purified by flash chromatography eluting with 1:3 EtOAc/hexanes to yield the title compound.

### Example 4

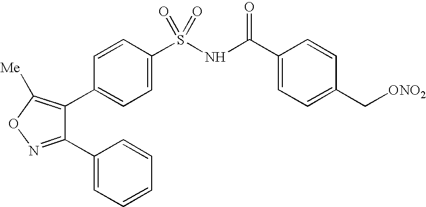

### 4-[({[4-(5-METHYL-3-PHENYLISOXAZOL-4-YL)PHENYL]SULFONYL}AMINO)CARBONYL]BENZYL NITRATE

Starting from valdecoxib, the title compound is prepared by following the same conditions as described in Example 3.

### Example 5

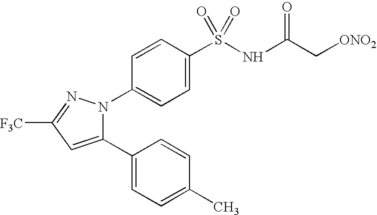

### 2-[({4-[5-(4-METHYLPHENYL)-3-(TRIFLUOROMETHYL)-1H-PYRAZOL-1-YL]PHENYL}SULFONYL)AMINO]-2-OXOETHYL NITRATE

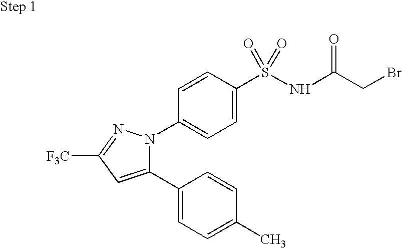

### N-(Bromoacetyl)-4-[5-(4-methylphenyl)-3-(trifluoromethyl)-1H-pyrazol-1-yl]benzenesulfonamide

To a solution of Celebrex (1.9 g, 5 mmol) and Et₃N (0.7 g, 7 mmol) in 30 ml of CH₂Cl₂ is added 2-bromoacetyl bromide (1.0 g, 5 mmol) at 0° C. After 1 h of stirring at rt, the reaction mixture is poured into a separatory funnel containing EtOAc(50 mL)/NH₄Cl(50 mL, sat). The phases are separated and the aqueous phase is extracted twice with EtOAc (2×50 mL). The organic layers are combined, washed with brine, dried over anhydrous Na₂SO₄ and evaporated to dryness. The crude material can be further purified by flash chromatography eluting with 1:3 EtOAc/hexanes to yield the title compound.

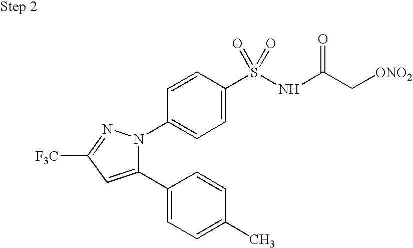

### 2-[({4-[5-(4-Methylphenyl)-3-(trifluoromethyl)-1h-pyrazol-1-yl]phenyl}sulfonyl)amino]-2-oxoethyl nitrate

To a solution of the product of Step 1 in 40 mL of acetonitrile is added AgNO₃ (1.7 g, 10 mmol) at room temperature. The reaction mixture is stirred for 30 min and then diluted with 100 mL of 1:1 EtOAc/hexane. The resulting suspension is filtered through a pad of silica gel and the filtrate is concentrated and the residue is purified by silica gel chromatography eluted with 2:1 hexane/EtOAc to give the title compound.

### Example 6

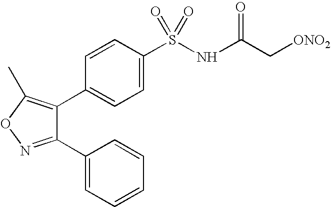

### 2-({[4-(5-METHYL-3-PHENYLISOXAZOL-4-YL)PHENYL]SULFONYL}AMINO)-2-OXOETHYL NITRATE

Starting from valdecoxib, the title compound is prepared by following the same conditions as described in Example 5.

### Example 7

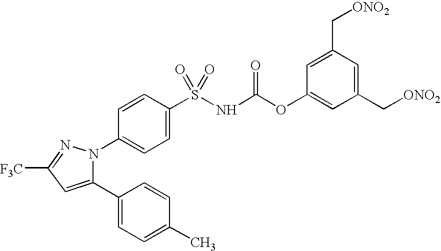

### 3,5-BIS[(NITROOXY)METHYL]PHENYL{4-[5-(4-METHYLPHENYL)-3-(TRIFLUOROMETHYL)-1H-PYRAZOL-1-YL]PHENYL}SULFONYLCARBAMATE

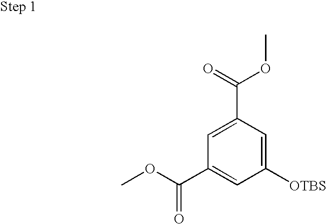

### Dimethyl 5-{[tert-butyl(dimethyl)silyl]oxy}isophthalate

A solution of 14.8 g of dimethyl 5-hydroxyisophthalate, 11.7 g of tert-butyldimethylsilyl chloride and 5.76 g of imidazole in 125 ml of DMF was stirred at 25° C. overnight under N₂. To quench the reaction, 300 ml of NH₄Cl sat and 300 ml of ethyl acetate were added and the mixture was stirred 10 min. The phases were separated and the aqueous phase was extracted twice with 100 ml of ethyl acetate. The organic layers were combined, washed with brine, dried over anhydrous Na₂SO₄, filtered and concentrated to yield 21.9 g of the title compound as a white powder. The crude material was used directly for the next step without further purification.

¹H NMR (acetone-d₆) δ 8.22 (s, 1H), 7.67 (s, 2H), 3.91 (s, 6H), 0.99 (s, 9H), 0.26 (s, 6H).

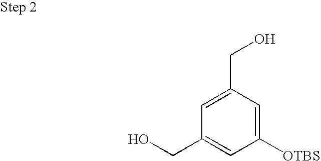

### 3-(tert-Butyl(dimehtyl)silyloxy)-5-hydroxymethylphenyl methanol

To a solution of 10.2 g of the previous compound in 100 mL of anhydrous THF under N₂ at −10° C. was added 135 mL of DIBAL-H (1M/hexanes) via a dropping funnel. The reaction was slowly warm-up to room temperature and stirred 3 h. To quench the reaction, 500 ml of Rochelle's salt and 300 ml of ethyl acetate were added and the mixture was stirred overnight. The phases were separated and the aqueous phase was extracted twice with 300 ml of ethyl acetate. The organic layers were combined, washed with brine, dried over anhydrous Na₂SO₄, filtered and concentrated to yield 8.37 g of the title compound as a white powder. The crude material was used directly for the next step without further purification.

¹H NMR (acetone-d₆) δ 6.90 (s, 1H), 6.74 (s, 2H), 4.55 (d, 4H), 4.12 (t, 2H), 0.99 (s, 9H), 0.20 (s, 6H).

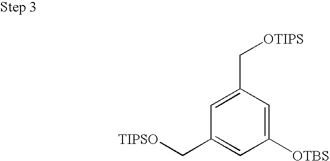

### 1-(tert-Butyl(dimethyl)silyloxy)-3,5-bis-triisopropylsilyloxymethyl benzene

A solution of 7.0 g of the previous compound, 12.3 mL of triisopropylsilyl chloride and 4.25 g of imidazole in 100 ml of DMF was stirred at 25° C. overnight under N₂. To quench the reaction, 300 ml of NH₄Cl sat and 300 ml of ethyl acetate were added and the mixture was stirred 10 min. The phases were separated and the aqueous phase was extracted twice with 100 ml of ethyl acetate. The organic layers were combined, washed with brine, dried over anhydrous Na₂SO₄, filtered and concentrated to yield 13.9 g of the title compound as a colorless oil. The crude material was used directly for the next step without further purification

¹H NMR (acetone-d₆) δ 7.01 (s, 1H), 6.83 (s, 2H), 4.85 (s, 4H), 1.22 (bs, 6H), 1.12 (d, 36H), 0.99 (s, 9H), 0.22 (s, 6H).

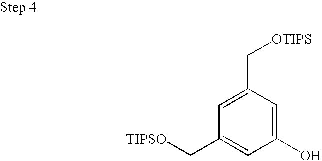

### 3,5-Bis-triisopropylsilyloxymethyl phenol

To a solution of 12.7 g of the previous compound in 120 ml of THF was added 22.2 mL of TBAF (1M/THF) at 0° C. under N₂. The reaction was stirred 15 min then quenched by adding 200 ml of NH₄Cl sat and 300 ml of diethyl ether were added and the mixture was stirred 15 min. The phases were separated and the aqueous phase was extracted twice with 100 ml of diethyl ether. The organic layers were combined, washed with brine, dried over anhydrous Na₂SO₄, filtered and concentrated to yield 9.65 g of the title compound as a colorless oil. The crude material was used directly for the next step without further purification

¹H NMR (acetone-d₆) δ 8.27 (s, 1H), 6.90 (s, 1H), 6.78 (s, 2H), 4.82 (s, 4H), 1.22 (bs, 6H), 1.12 (d, 36H).

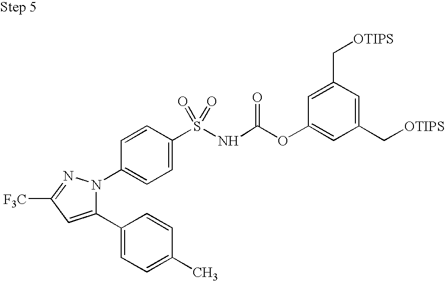

### 3,5-Bis[(triisopropylsilyloxy)methyl]phenyl {4-[5-(4-methylphenyl)-3-(trifluoromethyl)-1h-pyrazol-1-yl]phenyl}sulfonylcarbamate

To a solution of the previous phenol (product of Step 4) and triphosgene in CH₂Cl₂, pyridine is added dropwise at −78° C. After 1 h of stirring at rt, the final suspension is transferred via a canula to a solution Celebrex and pyridine mL CH₂Cl₂ cooled at −78° C. After 1 h at 0° C., the reaction mixture is poured into a separatory funnel containing EtOAc/NH₄Cl(sat). The phases were separated and the aqueous phase is extracted twice with EtOAc. The organic layers are combined, washed with brine, dried over anhydrous Na₂SO₄ and evaporated to dryness. The crude material is further purified by flash chromatography eluting with EtOAc/hexanes to yield the title compound.

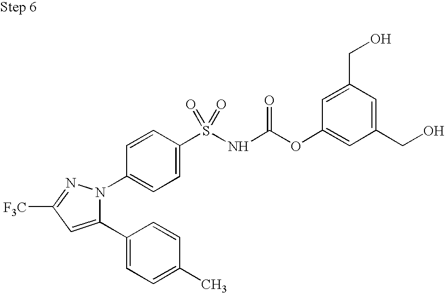

### 3,5-Bis(hydroxymethyl)phenyl {4-[5-(4-methylphenyl)-3-(trifluoromethyl)-1h-pyrazol-1-yl]phenyl}sulfonylcarbamate

A solution of the product of Step 5 at rt in acetonitrile under a nitrogen flow is added HF/pyridine complex (70% HF) over 5 min. After 1 h at room temperature, the reaction mixture is poured into a separatory funnel containing EtOAc/CuSO₄(sat). The phases are separated and the aqueous phase is extracted twice with EtOAc. The organic layers are combined, washed with CuSO₄(sat), brine, dried over anhydrous Na₂SO₄ and evaporated to dryness. The crude material is further purified by flash chromatography eluting with EtOAc/hexanes to yield the title compound.

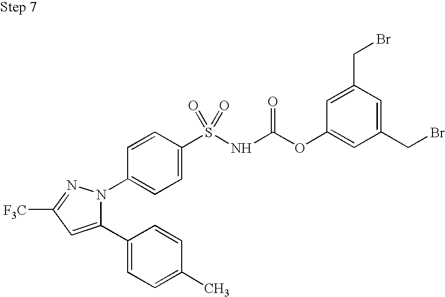

### 3,5-Bis(bromomethyl)phenyl{4-[5-(4-methylphenyl)-3-(trifluoromethyl)-1H-pyrazol-1-yl]phenyl}sulfonylcarbamate

To a solution of triphenylphosphine in CH₂Cl₂ is added bromine (1M/CH₂Cl₂) at 0° C. After 10 min, Hünig's base is added followed by the previous alcohol in CH₂Cl₂. After warming to room temperature, the reaction mixture is poured into a separatory funnel containing EtOAc/brine. The phases are separated and the organic layer is dried over anhydrous Na₂SO₄ and evaporated to dryness. The crude material is further purified by flash chromatography eluting with EtOAc/hexanes to yield the title compound.

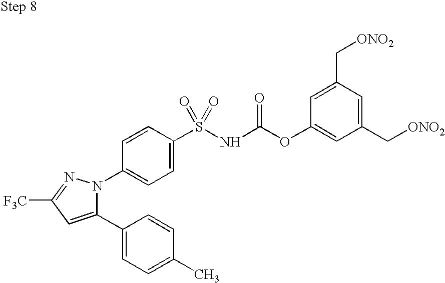

### 3,5-Bis[(nitrooxy)methyl]phenyl{4-[5-(4-methylphenyl)-3-(trifluoromethyl)-1h-pyrazol-1-yl]phenyl}sulfonylcarbamate

To a solution of the product of Step 7 in acetonitrile silver nitrate is added. After stirring for 2 h at room temperature the reaction mixture is diluted with 1:1 EtOAC/hexane and filtered through a pad of silica gel eluting with EtOAc. The solvents were removed under vacuo. The crude material was further purified by flash chromatography eluting with EtOAc/hexanes to yield the title compound.

### Example 8

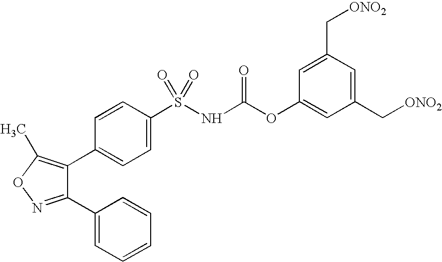

### 3,5-BIS[(NITROOXY)METHYL]PHENYL[4-(5-METHYL-3-PHENYLISOXAZOL-4-YL)PHENYL]SULFONYLCARBAMATE

Starting from valdecoxib, the title compound is prepared by following the same conditions as described in Step 5-8 of Example 7.

### Example 9

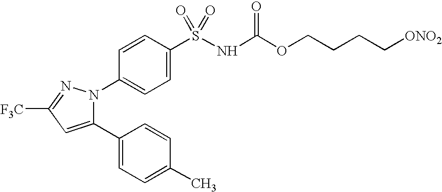

### 4-(NITROOXY)BUTYL{4-[5-(4-METHYLPHENYL)-3-(TRIFLUOROMETHYL)-1H-PYRAZOL-1-YL]PHENYL}SULFONYLCARBAMATE

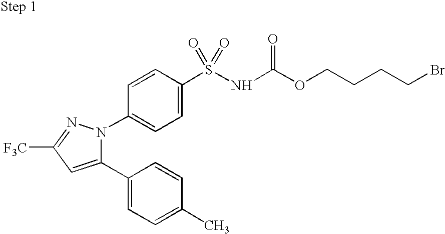

### 4-Bromobutyl{4-[5-(4-methylphenyl)-3-(trifluoromethyl)-1H-pyrazol-1-yl]phenyl}sulfonylcarbamate

To a solution of 4-bromobutanol (1.53 g, 10 mmol) and triphosgene (0.91 g, 3 mmol) in 100 ml of CH₂Cl₂, pyridine (0.86 g, 11 mmol) is added dropwise at −78° C. After 1 h of stirring at rt, the final suspension is transferred via a canula to a solution of Celebrex (3.81 g, 10 mmol) and pyridine 0.86 g in CH₂Cl₂ cooled at −78° C. After 15 min at 0° C., the reaction mixture is poured into a separatory funnel containing EtOAc(50 mL)/NH₄Cl(50 mL, sat). The phases are separated and the aqueous phase is extracted twice with EtOAc (2×50 mL). The organic layers are combined, washed with brine, dried over anhydrous Na₂SO₄ and evaporated to dryness. The crude material can be further purified by flash chromatography eluting with 1:3 EtOAc/hexanes to yield the title compound.

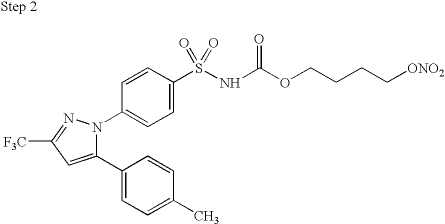

### 4-(Nitrooxy)butyl{4-[5-(4-methylphenyl)-3-(trifluoromethyl)-1h-pyrazol-1-yl]phenyl}sulfonylcarbamate

To a solution of the product of Step 1 in 40 mL of acetonitrile is added AgNO₃ (1.9 g, 20 mmol) at room temperature. The reaction mixture is stirred for 30 min and then diluted with 100 mL of 1:1 EtOAc/hexane. The resulting suspension is filtered through a pad of silica gel and the filtrate is concentrated and the residue is purified by silica gel chromatography eluted with 2:1 hexane/EtOAc to give the title compound.

### Example 10

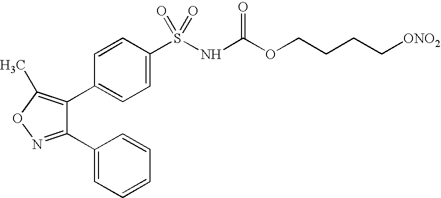

### 4-(NITROOXY)BUTYL[4-(5-METHYL-3-PHENYLISOXAZOL-4-YL)PHENYL]SULFONYLCARBAMATE

Starting from valdecoxib, the title compound is prepared by following the same conditions as described in Step 1-2 of Example 9.

1. A compound of Formula I or Formula II 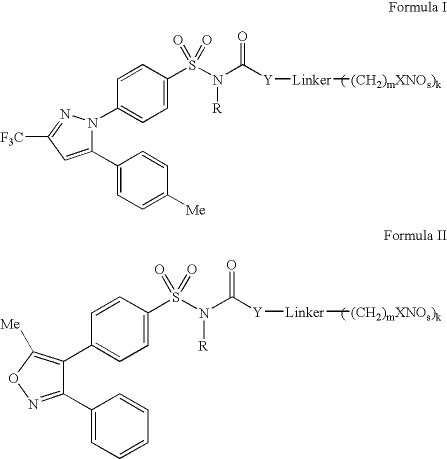
or a pharmaceutically acceptable salt thereof wherein
each s is independently 1 or 2;
k is 1, 2, 3 or 4;
each m is independently 0, 1, 2, 3 or 4;
each X is independently O or S;
Y is a bond, S, O or NR₁, wherein R1 is hydrogen or C₁₋₆alkyl;
R is hydrogen or C₁₋₆alkyl;
the linker is selected from the group consisting of:
(a) —(CH₂)ₙ, wherein n is 0, 1, 2, 3 or 4,
(b) C₃₋₆cycloalkyl, wherein the C₃₋₆cycloalkyl optionally mono-, di- or tri-substituted with a substituent selected from the group consisting of (1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(c) aryl, wherein the aryl is selected from the group consisting of phenyl and naphthyl, wherein the aryl is optionally mono-, di- or tri-substituted with a substituent selected from the group consisting of
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio,
(c) Heteroaryl optionally mono-, di- or tri-substituted with substituents selected from the group consisting of,
(1) halo,
(2) C₁₋₃alkyl,
(3) C₁₋₃alkoxy,
(4) Hydroxy,
(5) NO₂,
(6) CO₂,
(7) CF₃,
(8) CN;
(9) CH₂COOH
(10) CH₂COO—C₁₋₃alkyl,
(11) C₁₋₃alkthio.

2. The compound according to claim 1 wherein
s is 2;
k is 1;
m is 1 or 2.

3. The compound according to claim 1 wherein
X is O;
R is H.

4. The compound according to claim 1 wherein
R is H; and
Y is a bond.

5. The compound according to claim 1 wherein
R is H;
Y is a bond;
s is 2;
k is 1;
m is 1.

6. The compound according to claim 2 wherein:
the Linker is —(CH₂)ₙ, wherein n is 1 or 2.

7. The compound according to claim 2 wherein:
the Linker is C₃₋₆cycloalkyl, wherein the C₃₋₆cycloalkyl optionally mono-, di- or tri-substituted with a substituent selected from the group consisting of
(1) halo,
(2) Methyl,
(3) Methoxy,
(4) Hydroxy,
(5) NO₂,
(6) CO₂,
(7) CF₃,
(8) CN, and
(9) CH₂COOH.

8. The compound according to claim 2 wherein:
the Linker is aryl, wherein the aryl is selected from the group consisting of phenyl and naphthyl, wherein the aryl is optionally mono-, di- or tri-substituted with a substituent selected from the group consisting of
(1) halo,
(2) Methyl,
(3) Methoxy,
(4) Hydroxy,
(5) NO₂,
(6) CO₂,
(7) CF₃,
(8) CN, and
(9) CH₂COOH.

9. The compound according to claim 2 wherein:
the Linker is phenyl optionally mono-, di- or tri-substituted with a substituent selected from the group consisting of:
(1) halo,
(2) Methyl,
(3) Methoxy,
(4) Hydroxy,
(5) NO₂,
(6) CO₂,
(7) CF₃,
(8) CN, and
(9) CH₂COOH.

10. The compound according to claim 2 wherein:
the Linker is benzimidazolyl, benzofuranyl, benzopyrazolyl, benzotriazolyl, benzothiophenyl, benzoxazolyl, carbazolyl, carbolinyl, cinnolinyl, furanyl, imidazolyl, indolinyl, indolyl, indolazinyl, indazolyl, isobenzofuranyl, isoindolyl, isoquinolyl, isothiazolyl, isoxazolyl, naphthyridinyl, oxadiazolyl, oxazolyl, pyrazinyl, pyrazolyl, pyridopyridinyl, pyridazinyl, pyridyl, pyrimidyl, pyrrolyl, quinazolinyl, quinolyl, quinoxalinyl, thiadiazolyl, thiazolyl, thienyl, triazolyl, azetidinyl, 1,4-dioxanyl, hexahydroazepinyl, piperazinyl, piperidinyl, pyrrolidinyl, morpholinyl, thiomorpholinyl, dihydrobenzimidazolyl, dihydrobenzofuranyl, dihydrobenzothiophenyl, dihydrobenzoxazolyl, dihydrofuranyl, dihydroimidazolyl, dihydroindolyl, dihydroisooxazolyl, dihydroisothiazolyl, dihydrooxadiazolyl, dihydrooxazolyl, dihydropyrazinyl, dihydropyrazolyl, dihydropyridinyl, dihydropyrimidinyl, dihydropyrrolyl, dihydroquinolinyl, dihydrotetrazolyl, dihydrothiadiazolyl, dihydrothiazolyl, dihydrothienyl, dihydrotriazolyl, dihydroazetidinyl, methylenedioxybenzoyl, tetrahydrofuranyl, and tetrahydrothienyl, optionally mono-, di- or tri-substituted with substituents selected from the group consisting of,
(1) halo,
(2) Methyl,
(3) Methoxy,
(4) Hydroxy,
(5) NO₂,
(6) CO₂,
(7) CF₃,
(8) CN, and
(9) CH₂COOH.

11. The compound according to claim 10 wherein the Linker is selected from pyidyl, each optionally mono-, di- or tri-substituted with substituents selected from the group consisting of,
(1) halo,
(2) Methyl,
(3) Methoxy,
(4) Hydroxy,
(5) NO₂,
(6) CO₂,
(7) CF₃,
(8) CN, and
(9) CH₂COOH.

12. The compound according to claim 1 wherein s is 2.

13. A method of treating an inflammatory disease susceptible to treatment with a non-steroidal anti-inflammatory agent comprising administering to a patient in need of such treatment of a non-toxic therapeutically effective amount of a compound according to claim 1.

14. The method according to claim 13 wherein the patient is also at risk of a thrombotic cardiovascular event.

15. A method of treating cyclooxygenase mediated diseases advantageously treated by an active agent that selectively inhibits COX-2 in preference to COX-1 comprising administering to a patient in need of such treatment of a non-toxic therapeutically effective amount of a compound according to claim 1.

16. (canceled)

17. A method for treating a chronic cyclooxygenase-2 mediated disease or condition and reducing the risk of a thrombotic cardiovascular event in a human patient in need of such treatment and at risk of a thrombotic cardiovascular event comprising orally concomitantly or sequentially administering to said patient a compound according to claim 1 in an amount effective to treat the cyclooxygenase-2 mediated disease or condition and aspirin in an amount effective to reduce the risk of the thrombotic cardiovascular event.

18-24. (canceled)

25. A pharmaceutical composition comprising a compound according to claim 1 and aspirin in combination with a pharmaceutically acceptable carrier.

26. A pharmaceutical composition comprising a compound according to claim 1 and a pharmaceutically acceptable carrier.

27. A compound selected from the following group:

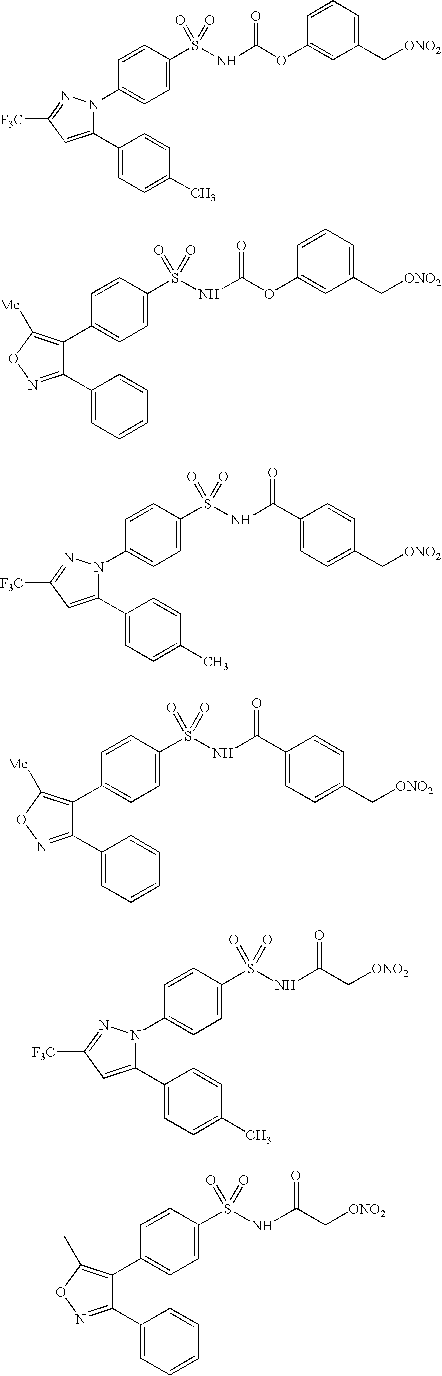

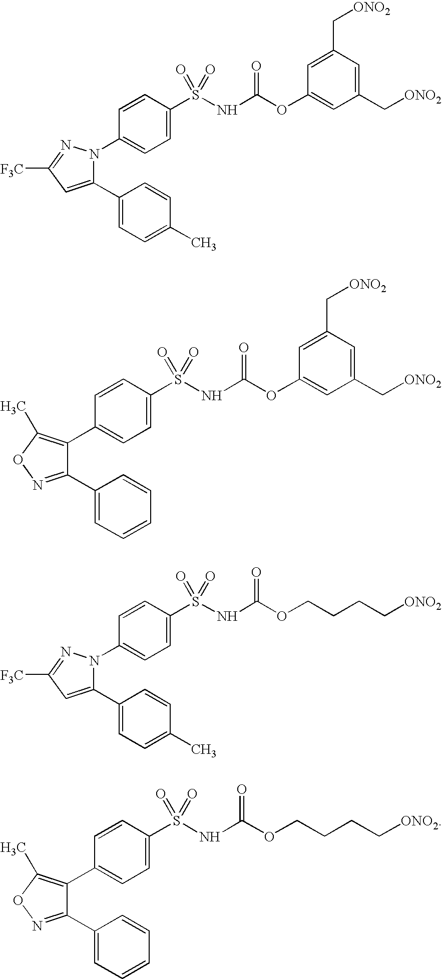

28. A compound of Formula I or Formula II 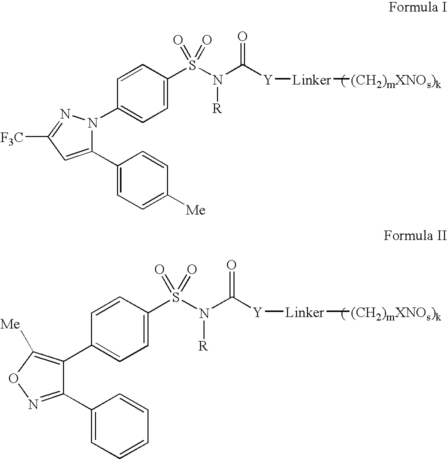
or a pharmaceutically acceptable salt thereof wherein
each s is independently 1 or 2;
k is 1, 2, 3 or 4;
each m is independently 0, 1, 2, 3 or 4;
each X is independently O or S;
Y is a bond, S, O or NR₁, wherein R1 is hydrogen or C₁₋₆alkyl;
R is hydrogen or C₁₋₆alkyl;
the Linker is selected from the group consisting of: [chemical structure]
